(19) United States
(12) Patent Application Publication
(10) Pub. No.: US 2005/0209608 A1
(43) Pub. Date: Sep. 22, 2005

(54) Trocar seal

(76) Inventor: Peter T. O'Heeron, Houston, TX (US)

Correspondence Address: Clarence E. Eriksen;Jackson Walker L.L.P., Suite 2100, 112 E. Pecan Street, San Antonio, TX, 78205-1521, US

(21) Appl. No.: 10/805,974

(22) Filed: Mar. 22, 2004

(51) Int. Cl. A61F 11/00

(52) U.S. Cl. 606/108

(57) ABSTRACT

A seal is disclosed for installation on the proximal end of a trocar to seal on surgical instruments having varying diameters. The seal has a general hourglass shape with an upper portion, a bottom portion and a sealing portion interposed between the upper and bottom portions. The bottom portion is formed for mating engagement with the access port at the proximal end of the trocar. A passage exists between the upper and bottom portions, and the diameter is expandable from 2 mm to about 13 mm. Various configurations of such a seal are disclosed.

### BACKGROUND OF THE INVENTION

1. Field of the Invention

The present invention relates to surgical instruments known as trocars which are used in endoscopic surgery to pierce or puncture an anatomical cavity to provide communication with the cavity during a surgical procedure. More particularly, the present invention relates to a seal to prevent the escape of insufflated gas during the performance of surgical procedures using the trocar.

2. Description of the Prior Art

Endoscopic surgery is an essential method of performing surgical operations and has become the surgical procedure of choice, because of its patient care advantages over “open surgery.” One form of endoscopic surgery is laparoscopic surgery, and a significant advantage of laparoscopic surgery over open surgery is the decreased post-operative recovery time. In most instances, a patient is able to leave the hospital within hours after laparoscopic surgery has been performed. With open surgery, several days of hospital care may be required before the patient is discharged. Additionally, laparoscopic surgery achieves decreased incidents of post-operative abdominal adhesions, decreased post-operative pain, and enhanced cosmetic results.

Conventionally, a laparoscopic surgical procedure begins with the insufflation of the abdominal cavity with carbon dioxide. The introduction of this gas into the abdominal cavity lifts the abdominal wall away from the internal viscera. The abdominal wall is then penetrated with a device known as a trocar, which includes a housing assembly, a cannula assembly attached to the housing assembly to form a bore through the trocar, and a piercing element called an obturator. The obturator slides through an access port formed on the upper (i.e. proximal) end of the housing assembly and through the bore of the trocar. The obturator has a diameter which is substantially the same as the diameter of the access port. After insertion of the trocar through the abdominal wall of the patient, the obturator is removed by the surgeon while leaving the cannula or tube protruding through the body wall. Laparoscopic instruments can then be inserted through the cannula to view internal organs and to perform surgical procedures.

Once the obturator is removed from the bore of the housing, it is necessary to obstruct the access port so that the carbon dioxide gas introduced into the abdominal cavity of the patient is contained. Traditionally, a trocar includes a spring-loaded flapper valve which opens when the obturator is inserted and which closes when the obturator is removed from the cannula to keep the insufflated gas from escaping. However, the insertion of laparoscopic instruments into the trocar re-opens the flapper door. To prevent escape of the insufflated gas upon insertion of a laporascopic instrument, a trocar also comprises a seal which is capable of providing sealing for laporascopic instruments having varying diameters e.g. between 5 mm and 12 mm. Since such seals are capable of providing sealing during the same surgical procedure for laporascopic instruments of varying diameters, they are commonly referred to as “universal” seals.

Various designs of universal seals have been proposed. See for example, U.S. Pat. Nos. 5,350,364; 5,385,553; 5,407,433; 5,512,053; 5,628,732, 5,827,228; 5,342,315; and 4,112,932 . Such prior art seals comprise a plurality of mechanical parts which must be assembled and are usually expensive.

### SUMMARY OF THE INVENTION

In accordance with the present invention, a seal is provided for installation on a trocar having a proximal end with an access port at the proximal end. The seal has a general hourglass shape with an upper portion and a bottom portion and a sealing portion interposed between the upper portion and the bottom portion. The bottom portion of the seal is formed for mating engagement with the access port at the proximal end of the trocar. The seal has a passage through it to permit a surgical instrument to pass through the seal.

In one embodiment, the diameter of the passage through the seal is approximately two millimeters in diameter. That diameter is expandable to about thirteen millimeters. Thus, a seal in accordance with the present invention is capable of providing the sealing function for surgical instruments of varying diameters between approximately two millimeters and thirteen millimeters.

A seal in accordance with the present invention may be fabricated from any suitable pliable material using a molding process, and is preferably fabricated from silicone. The seal is preferably coated with paralene or another approved coating.

In one embodiment, the sealing portion of a seal in accordance with the present invention comprises a plurality of corrugations of substantially uniform diameter. In a second embodiment, the sealing portion comprises a plurality of corrugations whose exterior diameters are generally uniform and whose interior diameters decrease from the upper portion of the seal to the bottom portion. In another embodiment, the sealing portion comprises a plurality of corrugations having decreasing diameters from the upper portion of the seal to the bottom portion of the seal. In yet another embodiment, the sealing portion comprises a plurality of corrugations having increasing diameters from the upper portion of the seal to the bottom portion. In yet another embodiment, the sealing portion of the seal has a spiral configuration, and in a fourth embodiment, the sealing portion is substantially smooth and of uniform diameter.

A trocar having either of the foregoing described seals constitutes an improvement over the prior art.

### BRIEF DESCRIPTION OF THE DRAWINGS

FIG. 1 is an exploded perspective view of an exemplary embodiment of a trocar.

FIG. 2 is a lateral section view of the proximal end of the body of the trocar shown in FIG. 1.

FIG. 3A is a perspective view of one embodiment of a trocar seal in accordance with the present invention.

FIG. 3B is a lateral section view of the trocar seal FIG. 3A and the proximal end of the trocar of FIG. 1 with the trocar seal installed.

FIG. 4A is a perspective view of another embodiment of a seal in accordance with the present invention.

FIG. 4B is a lateral section view of the trocar seal of FIG. 4A and the proximal end of the trocar of FIG. 1.

FIG. 4C is a lateral section view of an alternative embodiment of the seal of FIG. 4A.

FIG. 5A is a perspective view of another embodiment of a trocar seal in accordance with the present invention.

FIG. 5B is a lateral section view of the trocar seal of 5A and the proximal end of the trocar of FIG. 1 with the trocar seal installed.

FIG. 5C is a lateral section view of an alternative embodiment of the seal of FIG. 5A.

FIG. 6A is a perspective view of another embodiment of a trocar seal in accordance with the present invention.

FIG. 6B is a lateral section view of the trocar seal of FIG. 6A and the proximal end of the trocar of FIG. 1 with the trocar seal installed.

### DESCRIPTION OF THE PREFERRED EMBODIMENTS

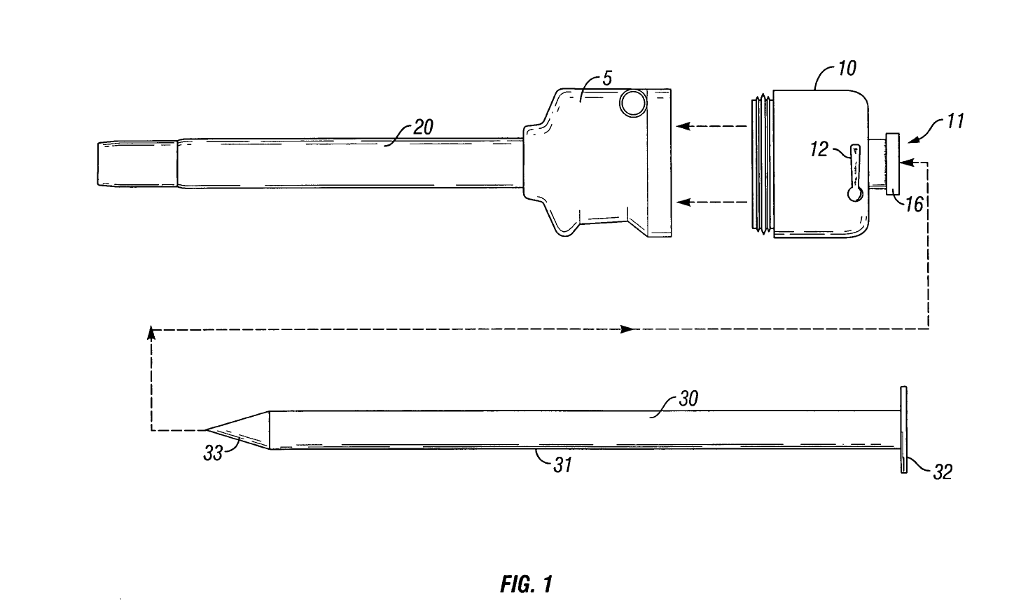

With reference to FIG. 1, an embodiment of a trocar 5 comprises a housing assembly 10 to which is attached a cannula assembly 20. The cannula assembly 20 is a hollow tube, and when attached to the housing assembly 10, a bore is defined through the trocar 5. An access port 11 is formed in the proximal end of the housing assembly 10 such that the access port 11 and the bore defined by the cannula assembly 20 are axially aligned. The diameter of the access port 11 may, for example, be between 2 mm and 22 mm.

Still with reference to FIG. 1, the trocar 5 also includes an obturator assembly 30 having a shaft 31 with an arcuate-shaped cap 32 at the proximal end of the shaft and a piercing tip 33 at the distal end of the shaft. The obturator assembly 30 has a diameter substantially the same as the diameter of the access port, and the obturator is inserted into the housing assembly 10 through the access port 11. The obturator slides in the bore that is defined by the combination of housing assembly 10 and cannula assembly 20. The trocar 5 may comprise a safety shield 21, although the present invention is not limited to seals for trocars with safety shields.

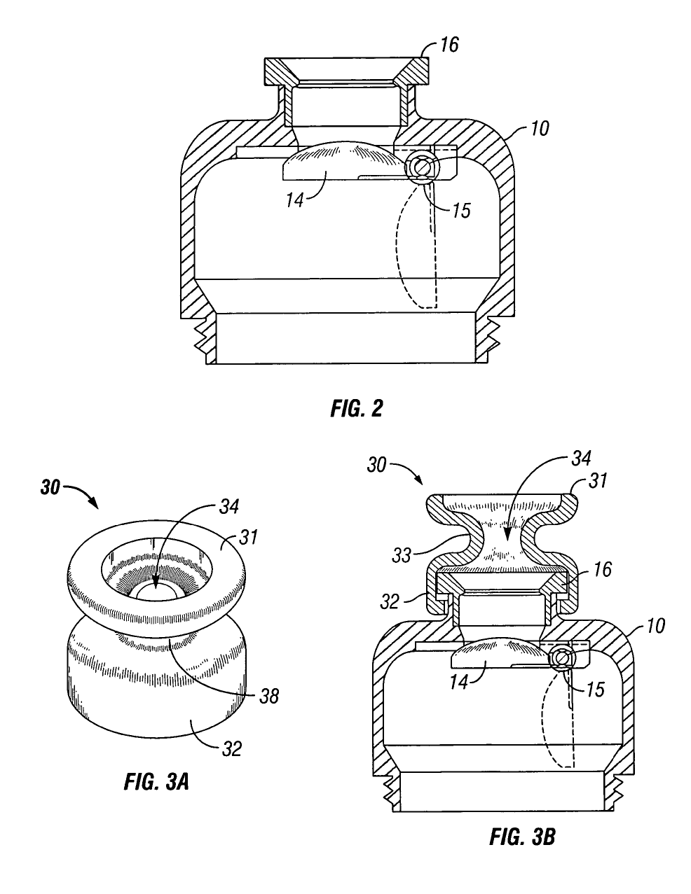

With reference to FIG. 2, the trocar 5 includes a flapper valve door 14 for regulating communication through the access port 11. As illustrated in FIG. 2, the flapper valve door 14 may have a domed protrusion 14A which engages the housing 10 to form a seal when the flapper valve door 14 is closed. The flapper valve door 14 is rotatably connected to the housing assembly 10, and the flapper valve door 14 is rotatable between a closed position and an open position, as shown by the dotted lines in FIG. 2. Resistance mechanisms, such as torsion springs or compression springs (not shown), may be used to bias the flapper valve door 14 in the closed position. A manual flapper door actuator 12 is provided for manual rotation of the flapper valve door 14 between the closed position and range of open positions. Withdrawal of the obturator assembly 30 from the housing assembly 10 results in the closure of the flapper valve door 14.

After the obturator assembly 30 is withdrawn from the trocar 5, a seal in accordance with the present invention is attached to access port 11, and a first embodiment of such a seal is illustrated in FIG. 3A. Seal 30 has a general hourglass shape with an upper portion 31 and a bottom portion 32. The bottom portion 32 is formed for mating engagement with the flange 16 at access port 11 at the proximal end of the trocar. Interposed between the upper portion 31 and bottom portion 32 is sealing portion 33. In the seal shown in FIGS. 3A and 3B, a passage 34 exists between the upper portion 31 and bottom portion 32 for insertion of a surgical instrument (not shown).

The unexpanded diameter of the passage 34 in seal 30 is about 2 mm and the diameter of the passage is expandable to about 13 mm without loss of sealing function. The interior of the sealing portion of seal 30 is smooth as shown in FIGS. 3A and 3B.

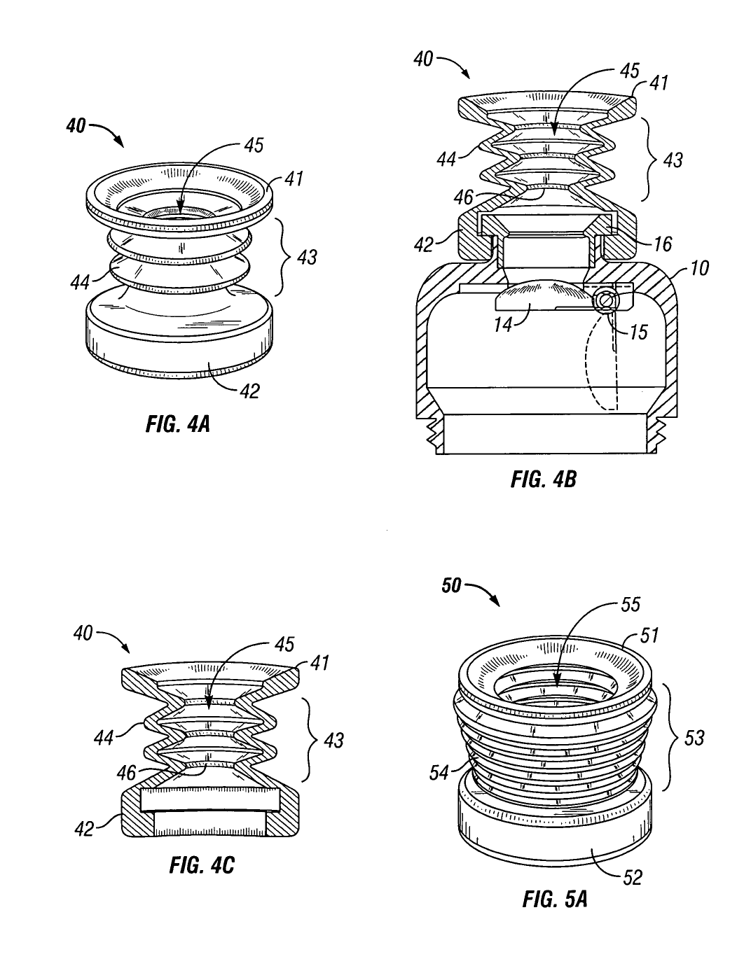

With reference now to FIGS. 4A and 4B, a second embodiment of a seal in accordance with the present invention is illustrated. Seal 40 has a general hourglass shape and has an upper portion 41 and a bottom portion 42. Interposed between the upper portion 41 and bottom portion 42 is a sealing portion 43, which in this embodiment comprises a plurality of corrugations 44. Corrugations 44 have a uniform outer diameter between the upper portion 41 and bottom portion 42 of seal 40. A passage 45 exists between the upper portion 41 and the bottom portion 42 for the insertion of a surgical instrument. In the embodiment of FIG. 4B, the inner diameters of the corrugations decrease from the upper portion of the seal to the bottom portion of the seal. Alternatively, the inner diameters of the corrugations may be uniform between the upper and bottom portions of seal 40 as shown in FIG. 4C. The unexpanded diameter of passage 45 at point 46 is 2 mm. The diameter of passage 45 is expandable to about 13 mm without loss of sealing function.

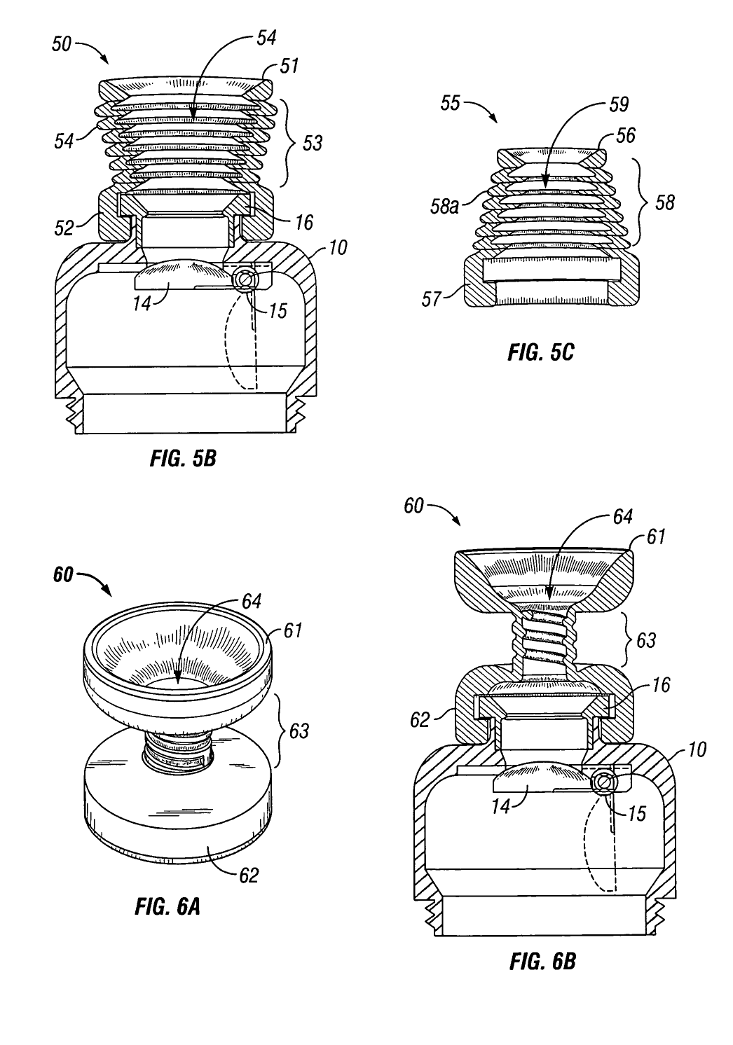

Referring now to FIGS. 5A and 5B, a third embodiment of a seal in accordance with the present invention is illustrated. Seal 50 has a general hourglass shape and comprises upper portion 51 and bottom portion 52. Sealing portion 53 is interposed between upper portion 51 and bottom portion 52. In this embodiment, sealing portion 53 comprises a plurality of corrugations 54, and the diameters of those corrugations decrease from the upper portion 51 toward the bottom portion 52 of seal 50. A passage 55 exists between the upper portion 51 and bottom portion 52 for the insertion of a surgical instrument, and the unexpanded diameter of the corrugations closest to bottom portion 52 is 2 mm. The diameter of passage 55 is expandable to about 13 mm without loss of sealing function.

Turning to FIG. 5C, an alternative embodiment of the seal shown in FIGS. 5A and 5B is illustrated. Seal 55 has a general hourglass shape and comprises an upper portion 56 and a bottom portion 57, with sealing portion 58 interposed between the upper and bottom portions. Sealing portions 58 comprise a plurality of corrugations 58a, which increase in diameter from the upper portion 56 to the bottom portion 57 of seal 55. The diameter of passage 59 through seal 55 is 2 mm in its unexpanded state and is expandable to 13 mm without loss of sealing function.

Now with reference to FIGS. 6A and 6B, a fourth embodiment of a seal in accordance with the present invention is shown. Seal 60 comprises an upper portion 61, a bottom portion 62, and a sealing portion 63 interposed between the upper and bottom portions. In this embodiment, the diameter of sealing portion 63 is substantially uniform between the upper portion 61 and the bottom portion 62, and sealing portion 63 comprises a spiral configuration 64. A passage 64 exists between the upper portion 61 and bottom portion 62 for the insertion of a surgical instrument. As in other embodiments, the unexpanded diameter of passage 55 is 2 mm, and that diameter is expandable to about 13 mm without loss of sealing function.

The bottom portions of seals 40, 58, and 60 are formed for mating engagement with flange 16 at the proximal end of the trocar.

All of the seals of 30, 40, 50, 55 and 60 are formed from a suitable pliable material using a molding process and silicone or other approved flexible rubber or plastic is the preferred material for the seal. The silicone material is also preferably coated with a paralene or other approved coating, so that a surgical instrument will not drag on the seal as it is inserted or withdrawn.

1. A seal for installation on a trocar having a proximal end that has an access port, which seal has: (i) a general hourglass exterior shape; (ii) a passage through the seal for insertion of a surgical instrument; (iii) an upper portion and a bottom portion, said bottom portion being formed for mating engagement with the access port; and (iv) a sealing portion interposed between the upper portion and the bottom portion.

2. The seal of claim 1, wherein the diameter of the passage through the seal is approximately two millimeters.

3. The seal of claim 2, wherein the diameter of the passage through the seal is expandable from approximately two millimeters to about thirteen millimeters.

4. The seal of claim 1, wherein the seal comprises a pliable material.

5. The seal of claim 4, wherein the pliable material is silicone.

6. The seal of claim 1, wherein the sealing portion of the seal comprises a plurality of corrugations, whose interior diameters are generally uniform and whose exterior diameters are generally uniform.

7. The seal of claim 6, wherein the diameter of the passage through the seal is approximately two millimeters.

8. The seal of claim 7, wherein the diameter of the passage through the seal is expandable from two millimeters to about thirteen millimeters.

9. The seal of claim 6, wherein the seal comprises a pliable material.

10. The seal of claim 9, wherein the pliable material is silicone.

11. The seal of claim 1, wherein the sealing portion of the seal comprises a plurality of corrugations that decrease in diameter from the upper portion of the seal to the bottom portion of the seal.

12. The seal of claim 11, wherein the diameter of the passage through the seal is approximately two millimeters.

13. The seal of claim 12, wherein the diameter of the passage through the seal is expandable from approximately two millimeters to about thirteen millimeters.

14. The seal of claim 11, wherein the seal comprises a pliable material.

15. The seal of claim 14, wherein the pliable material is silicone.

16. The seal of claim 1, wherein the sealing portion has a substantially smooth surface of uniform diameter.

17. The seal of claim 16, wherein the diameter of the passage through the seal is approximately two millimeters.

18. The seal of claim 17, wherein the diameter of the passage through the seal is expandable from approximately two millimeters to about thirteen millimeters.

19. The seal of claim 16, wherein the seal comprises a pliable material.

20. The seal of claim 19, wherein the pliable material is silicone.

21. The seal of claim 1, wherein the sealing portion of the seal comprises a plurality of corrugations that increase in diameter from the upper portion of the seal to the bottom portion of the seal.

22. The seal of claim 21, wherein the diameter of the passage through the seal is approximately 2 millimeters.

23. The seal of claim 22, wherein the diameter of the passage in the seal is expandable from 2 millimeters to about 13 millimeters.

24. The seal of claim 21, wherein the seal comprises pliable materials.

25. The seal of claim 24, wherein the pliable material is silicone.

26. The seal of claim 1, wherein the sealing portion of the seal comprises a plurality of corrugations where exterior diameters are generally uniform and whose interior diameters decrease in diameter from the upper portion of the seal to the bottom portion of the seal.

27. The seal of claim 26, wherein the diameter of the passage through the seal is approximately 2 millimeters.

28. The seal of claim 27, wherein the diameter of the passage in the seal is expandable from 2 millimeters to about 13 millimeters.

29. The seal of claim 26, wherein the seal comprises pliable materials.

30. The seal of claim 29, wherein the pliable material is silicone.
